(19) United States
(12) Patent Application Publication
(10) Pub. No.: US 2017/0245765 A1
(43) Pub. Date: Aug. 31, 2017

(54) DEVICE FOR MEASURING HUMAN HEARTBEAT RATE, RESPIRATION RATE AND BODY TEMPERATURE

(71) Applicants: Ding Yuan YEN, Kowloon (HK); Hsaio Wei FANG, New Taipei City (TW); Te Min LAI, Taichung (TW)

(72) Inventors: Ding Yuan YEN, Kowloon (HK); Hsaio Wei FANG, New Taipei City (TW); Te Min LAI, Taichung (TW)

(73) Assignees: Nelsen Yen, BREA, CA (US); Ding Yuan Yen, BREA, CA (US)

(21) Appl. No.: 15/438,135

(22) Filed: Feb. 21, 2017

(30) Foreign Application Priority Data
Feb. 26, 2016 (CN) 201620145063.0

(51) Int. Cl. A61B 5/0205

(52) U.S. Cl. CPC A61B 5/02055; A61B 5/015

(57) ABSTRACT

The utility model relates to a device for measuring the heartbeat, respiration, and body temperature of a human body, comprising of a shell and a base, wherein a PCB board, a buzzer, and a multi-point infrared temperature sensor and a camera are arranged in the shell. The infrared temperature sensor is used for temperature sampling of M×N sampling points, where M≧3, N≧3, and the camera is used for acquiring the video image of the measured object.

### CROSS-REFERENCE TO RELATED APPLICATIONS

This application claims priority of Chinese Patent Application No. 201620145063.0, filed on Feb. 26, 2016, the entire contents of which are hereby incorporated herein by reference.

### TECHNICAL FIELD

The present invention relates to a device for measuring human body heartbeat, respiration, and body temperature, in particular to a device for measuring human body temperature based on multi-point infrared temperature sensor and measuring heartbeat and breathing based on high-definition camera.

### BACKGROUND

Since human heartbeat rate, respiratory rate, and body temperature are important parameters of human life, accurate and convenient measurement of heartbeat rate, respiratory rate, and body temperature is of importance for evaluating physical health. The traditional methods of measuring the heartbeat rate of the human body having mainly been through pulse detection, the existing detection methods, including heart rate monitor and heart rate meter, require close contact of measuring devices with the measured person. The prior art (CN 101959458A) also discloses a method of measuring heartbeat rate using a visible light image by measuring a plurality of visible light images by receiving a plurality of visible light images corresponding to the skin of a subject near the blood-feeding capillary of the heart. However, the method still requires close contact with the skin of the measured object in order to obtain a clear capillary image, and the quality of image acquisition seriously affects the accuracy of the test.

In recent years, with improvements in infrared temperature measurement technology, non-contact infrared thermometers have experienced rapid development, better performance, and continuous improvement. Compared with the contact temperature measurement method, infrared temperature has benefits such as fast response time, is non-contact, may be safer, and others. Infrared detection is a kind of on-line monitoring type high-tech detection technology, which integrates photoelectric imaging technology, computer technology and image processing technology. It receives the infrared ray (infrared radiation) from the object and displays its thermal image on the screen with accuracy and speed. However, the standard existing infrared temperature measurement devices use a single point of measurement, which susceptible to ambient temperature interference and makes the error range large.

Therefore, achieving accurate heartbeat rate, respiratory rate, and body temperature measurement are technical problems to be solved urgently.

### SUMMARY

In order to meet people's demand for convenient measurement of heartbeat, respiration, and body temperature, the invention solves the technical problem that the traditional techniques face, namely inability to measure the human heart rate, respiratory rate and body temperature in a remote and accurate manner at the same time. The utility model provides an infrared temperature sensor and HD camera for measuring body temperature. The image acquired by the camera is processed and the position of the face and the chest is detected by the image recognition technology. The heartbeat and respiration are determined by the change of the face and the chest position, and then the heart rate and the respiratory rate are obtained, achieving long-range heartbeat and breathing measurements. The high precision infrared temperature sensor in the camera is used for multi-point temperature sampling of multiple sampling points on the measured object, and eliminates points with sampling temperature that are obviously not within the human body temperature range. The remaining sampled effective temperatures are averaged to obtain more accurate temperature data, which greatly reduces the interference of the ambient temperature and realizes real-time, fast, non-contact body temperature measurement.

The technical proposal for solving the technical problem of the invention is as follows:

An apparatus for measuring human heartbeat, respiration and body temperature comprises a case and a base, wherein a PCB board, a buzzer and a multi-point infrared temperature sensor and a camera are arranged in the shell; the multi-point infrared temperature The sensor is used for performing temperature sampling on a plurality M×N of sampling points, where M×3, N×3, and the camera is used for acquiring the video image of the measured object.

Further, a microphone and a reset switch are provided in the housing.

Further, a base is provided below the housing, and the housing and the base are connected by columns in an adjustable direction.

Further, a data line interface is provided below the rear portion of the housing.

Compared with the prior art, the utility model obtains the video image needed for analyzing the measured object through the camera, obtains the temperature for the measurement interval required for measuring the body temperature through the infrared temperature sensor, and connects to the cloud server through the data transmission. It avoids the shortcomings of the current measurement techniques for heartbeat rate and breathing. It also avoids the current temperature measurement techniques' flaw single point temperature susceptibility to environmental defects; and through effective temperature screening, via excluding temperatures outside the normal human body temperature range as may be factors such as clothing and abnormal sampling temperature effects, increases body temperature measurement accuracy. Therefore, it achieves accurate and accurate measurement of the human heartbeat, respiration and body temperature.

### BRIEF DESCRIPTION OF THE DRAWINGS

FIG. 1 is an enlarged view of the structure provided by the utility model.

FIG. 2 is a front schematic view of the utility model.

FIG. 3 is a back view of the utility model.

FIG. 4 is a schematic view of the measured body temperature provided by the utility model.

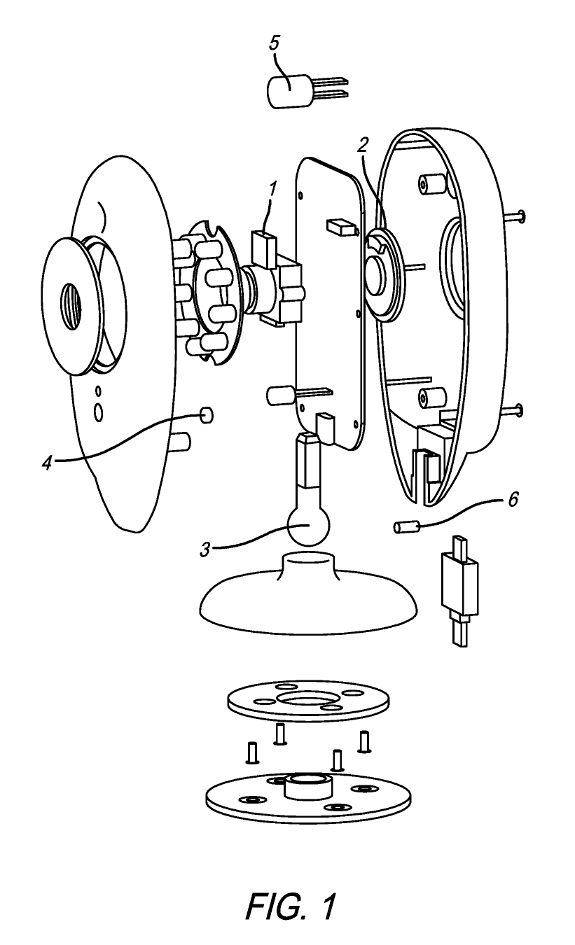

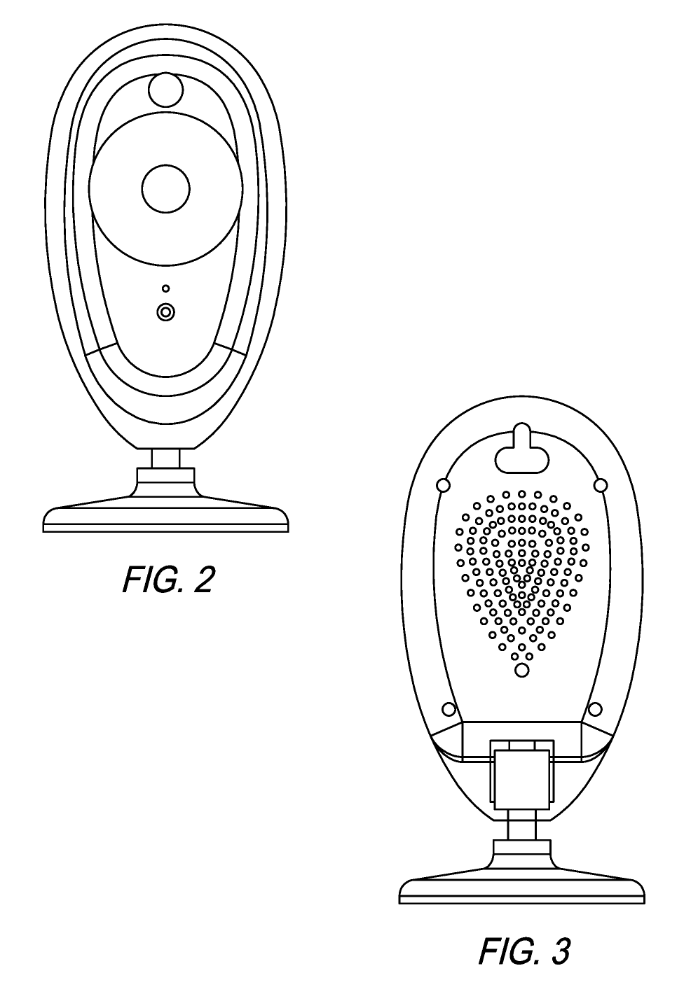

### DETAILED DESCRIPTION

In order to describe the structure and the technical solution of the present invention, the following description will be made in detail with reference to the accompanying drawings. The cited examples are for the purpose of explaining the present invention and are not intended to limit the scope of the utility model.

Heart rate is an important indicator of health. Each person's heart rate is different, with normal adult normal heart rate ranging around 60 to 100 times per minute. Normal heart rhythm is affected by many factors, when the movement of the heartbeat will speed up, rest or sleep when the heartbeat will slow down, exhaled heartbeat slower, fever, tension, excessive pressure, pain, etc., will also affect the heart rate. Heart rate is affected by breathing speed, under normal circumstances, women heart rate faster than men, normal adults breathing about 16-20 times per minute, and the heart rate is 1:4, that is, every breath, heart beat four times.

Heart beat blood flows through the contraction of the heart into the aorta, and is then passed to the systemic arteries. When the blood enters into the blood vessels of the head, the pulse will make the whole face fluctuate slightly. The amplitude of these fluctuations is quite subtle, under normal circumstances the human eye cannot directly detect these subtle changes, but through the high-speed camera shooting slowed down, any slight fluctuations can be accurately captured. The present invention mainly utilizes the fluctuation of the facial area of the body when the heartbeat occurs, and then forms a fine displacement in the image. By recognizing the small displacement of the facial area in the image frame, the heartbeat can be judged and then the heartbeat rate is counted and calculated. In addition, as the heart beats, facial blood vessels will be followed by congestion, facial color will have a small change, through the capture and analysis of the camera, you can change the frequency of face color to get heart rate. Respiratory frequency measurement principle and the principle of the heart rate measurement similar to the lung and the air exchange, when the lungs inhalation of air will make the chest ups and downs, by capturing the image of the chest between the site changes to identify the occurrence of breathing, and then analyze the respiratory rate. The foregoing is an explanation of the principles of the present invention, which will be further described below with reference to examples.

In order to realize the purpose of remote accurate measurement of human heart rate, respiratory rate and body temperature, as well as realizing sharing of cloud data transmission, the utility model sets an 8 million pixel high-definition camera 1, multi-point infrared temperature sensor 5, micro-microphone 4, buzzer 2, and an adjustable direction pillar 3 and a reset switch 6.

The utility model uses a high-definition camera 1 to continuously shoot the measured object to obtain a series of video images, and then analyzes the video images to obtain the heart rate and the respiratory rate. In one of these methods, a series of images were taken with 1080p at 30 frames per second using an 8-megapixel wide-angle lens. The image is extracted by multi-frame image processing, 1080P image resolution of 1920×1080 about two million frames, through the face recognition program to lock the face position of each image, using the face recognition algorithm to locate the face in each image. The faces of each image are located by the face recognition algorithm, and the position of the face in the image is determined by the upper left corner and the lower right corner of the frame. The image is illustrated with 30 frames per second for example. The coordinates of the upper left corner and lower right corner of the region are as follows:

(230,410)(540,660), (230,410)(540,660), (230,410)(540,660), (230,410)(540,660), (230,410)(540,660), (230,410)(540,660), (230,410)(540,660), (230,410)(540,660), (230,410)(540,660), (231,411)(541,661), (231,411)(541,661), (231,412)(541,662), (231,412)(541,662), (231,411)(541,661), (231,411)(541,661), (230,410)(540,660), (230,410)(540,660), (230,410)(540,660), (230,410)(540,660), (230,410)(540,660), (230,410)(540,660), (230,410)(540,660), (230,410)(540,660), (230,410)(540,660), (230,410)(540,660), (230,410)(540,660), 230,410)(540,660), (230,410)(540,660), (230,410)(540,660), (230,410)(540,660)

As can be seen from the above data, the measured object in the inertial oscillation direction is slightly to the top right (to the observer's point of view), from the beginning of the first 12 to 19 images for the emergence of a fluctuation, which represents a when a heartbeat occurs. Followed by analysis of the follow-up video frame, this can produce a count of the number of heartbeats that occur per minute, and thus the heart rate.

Breathing frequency measurement and heart rate measurement are similar. Extracted from the image of multi-frame image, face detection algorithm is used to determine the location of the face in each image; according to the location of the face, determine the location of the thoracic position; detect the chest region, a region located 1.5 to 2.0 face lengths below the upper edge of the face; near the location of the chest, identify the generation of breathing, respiratory ups and downs when an event is captured on behalf of a breathing occurs; according to the number of changes in chest location per minute, the respiratory rate can be obtained.

The measured human heart rate and respiratory rate can also be set up in real time and uploaded to the cloud server in the region. The user can bind the device account login and connect to the server for real-time view of the measured heart rate and respiration. Preferably, the invention further comprises a face recognition unit which records the historical heart rate and the respiratory rate data of each measured object. When it is found that the heart rate or the respiratory rate measured by the same subject has large differences from the historical measured data, this may suggest that physical condition of the measured object may be abnormal, which can be detected via remote monitoring and can trigger alerts to the users.

Normal body temperature is generally 36.1° C.˜37° C., lower than the oral temperature by about 0.2° C.˜0.4° C., according to the level of fever (oral temperature), can be divided into: low heat: 37.4° C.˜38° C.; 39° C. to 41° C.; ultra-high heat: 41° C. or higher; and in the present invention, the effective temperature range of the human body is set to 35° C. to 42° C., and points are only considered when the collected temperature falls within the above-mentioned effective temperature range. Otherwise, it means that the corresponding sampling points do not belong to the human body area or the human body area is blocked by other objects, resulting in abnormal temperature. By eliminating the above abnormal temperature, the average temperature of the sample obtained can produce more accurate body temperature data, avoiding the errors of single point measurement due to the impact of environmental interference. The above is an explanation of the principle of the present invention, and the following description will be given by way of example.

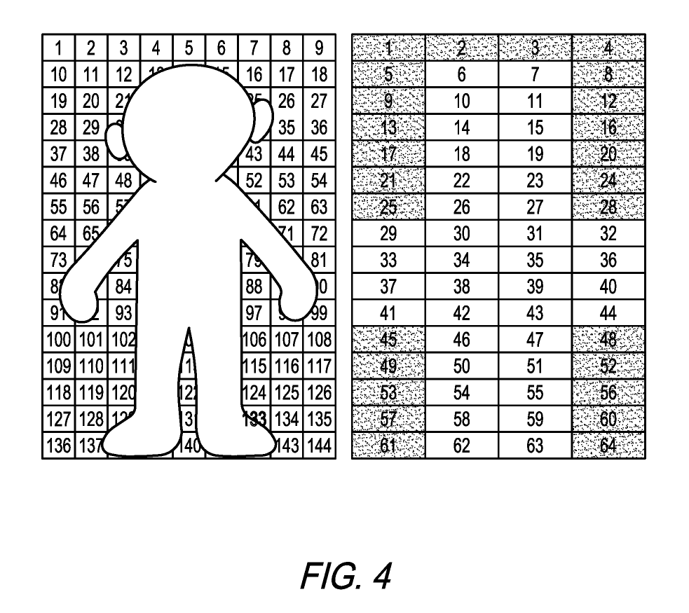

In the camera on the side set up a high-precision infrared temperature sensor 5, the infrared temperature sensor can simultaneously collect the temperature of 64 sampling points, the effective temperature acquisition distance of 1.5 meters or more, 64 sampling points in matrix distribution, as shown in FIG. 4; 64 sampling points are distributed in the whole imaging area. When the human body is detected in the camera shooting area, the infrared temperature sensor is triggered to carry out the temperature sampling, and the temperature corresponding to the 64 sampling points is obtained. Only some of the sampling points fall within the effective region of the human body. Because some of the sampling points are not covered by the human body, or because of object occlusion and other reasons, some of the sampling points of the temperature significantly deviate from the human body temperature range. In calculating the body temperature, abnormal points are removed from the sampling points. If the temperature of the sampling point is not within the effective temperature range of the human body, it is considered that the temperature is not the effective temperature and is not taken into account in the calculation; if the sampling point has temperature 35° C.˜42° C., then it is within the effective temperature rate. The temperatures of sampling points which belong to the effective temperature range are averaged, and the average temperature obtained is the measured body temperature.

The measured temperature data will be uploaded to the local cloud server in real time. Therefore, no matter where the user is, the user can simply open the browser to the specified page or login account through APP to view real-time camera image and body temperature data, remote body temperature measurement, and monitoring. Preferably, the invention further comprises a face recognition unit which records the historical temperature data of each measured object and when it is found that the body temperature measured by the same subject has a greater difference than the body temperature measured recently by the history, it will note that there may be physical anomalies and will be timely to alert the user about the noted temperature difference.

The above is only a few embodiments of the utility model, and is not intended to limit the scope of the utility model. Any modifications, equivalent replacements, improvements, and the like within the spirit and principle of the utility model shall be included in The protection scope of the utility model is within the scope.

What is claimed is:

1. A device for measuring the heartbeat, respiration, and body temperature of a human body, comprising of a shell and a base, wherein a PCB board, a buzzer, and a multi-point infrared temperature sensor and a camera are arranged in the shell. The infrared temperature sensor is used for temperature sampling of M×N sampling points, where M≧3, N≧3 , and the camera is used for acquiring the video image of the measured object.

2. The device for measuring the heart rate, respiration and body temperature of a human body according to claim 1, characterized in that a microphone and a reset switch are provided in the housing.

3. The device for measuring the heart rate, respiration, and body temperature of a human body according to claim 1, characterized in that a base is provided below the housing, and the housing and the base are connected by an adjustable column.

4. The device for measuring the heart rate, respiration, and body temperature of a human body according to claim 1, wherein a data line interface is arranged below the rear part of the housing.
